(19) United States
(12) Patent Application Publication
(10) Pub. No.: US 2021/0082019 A1
(43) Pub. Date: Mar. 18, 2021

(54) Customizable Platform for Ingredient Selectable Skin Treatments In Combination with a Packaging System for Mixing by an End User

(71) Applicants: Assif Versano, Raanana (IL); Joseph Abergel, Los Angeles, CA (US)

(72) Inventors: Assif Versano, Raanana (IL); Joseph Abergel, Los Angeles, CA (US)

(21) Appl. No.: 17/023,225

(22) Filed: Sep. 16, 2020

Related U.S. Application Data
(60) Provisional application No. 62/900,805, filed on Sep. 16, 2019.

(51) Int. Cl. G06Q 30/06; A61K 8/65; A61K 8/67; A61K 8/9794; A61K 8/92; A61K 8/73; A61K 8/9789; A61Q 19/00; G16H 10/20; G06Q 30/02; G06Q 10/08; G06Q 20/12; G16H 70/40; G16H 50/70; G06N 20/00

(52) U.S. Cl. CPC G06Q 30/0621; G16H 50/20; A61K 8/673; A61K 8/9794; A61K 8/671; A61K 8/678; A61K 8/676; A61K 8/922; A61K 8/735; A61K 8/9789; A61Q 19/00; G16H 10/20; G06Q 30/0203; G06Q 10/0832; G06Q 30/0631; G06Q 30/0205; G06Q 20/127; G16H 70/40; G16H 50/70; G06Q 30/0282; G06N 20/00; A61K 8/65

(57) ABSTRACT

A customizable platform for ingredient selectable skin treatments. End users purchase pre-configured kits, which provide pre-made combinations of active ingredients that are already grouped together. Next, end users configure their kits' active ingredients in the following ways: First an end user builds their own formulas with an active ingredients selection process, allowing the end user to specify the ingredients to include; Second, the end user completes a skin profile questionnaire where the end user answers a series of questions regarding their skin type and preferences. The platform analyzes user data to determine, create, and assemble the ideal skincare product for the end user. Skin care products are shipped in packaging that includes a base cream, active ingredients, and a spoon or other stirring device or apparatus for mixing. Upon receiving the kit, the end user mixes the cream together with the active ingredients to create a potent solution.

### TECHNICAL FIELD OF THE INVENTION

The present invention relates generally to customizable skin treatment compounds and mixtures. More specifically, the present invention relates to customizable skin treatment compounds and mixtures delivered to end users in a packaging system that keeps the individual ingredients separate until mixed by the end user.

### BACKGROUND OF THE INVENTION

Currently, the skin care and vitamin industry is struggling with several major shortcoming in their product offerings and delivery to the end consumer; product customization, freshness, and potency.

The quickly growing market of cosmetic products demands new formulations with new active ingredients while maintaining a prompt and effective product development life cycle. While products chemical and clinical tests may take significant time, cosmetic companies use mass approach for product development in order to guarantee the stability of physical appearance and safety. But at the same time data related to the chemical stability and potency of cosmetic products remains very scarce.

With respect to customization, there are a myriad of skin types and conditions that require varying treatments consisting of varying compositions and mixtures of ingredients to be effective. Pre-packaging of general skin treatment compositions and mixtures directed to the mass market have resulted in a compromise of product offerings, as well as strength and potency issues resulting from extended times from manufacture to purchase due to inventory storage and time spent sitting on the shelf and/or at storage before purchase and use.

Therefore, what is needed is a method for allowing custom ordering and manufacturing of skin treatment products that is manufactured and delivered with a quicker turnaround. Additionally, providing skin care treatment products in packaging that keeps the ingredients separate until mixed and activated by an end user and increases potency and freshness for the end user with ease of use.

### SUMMARY OF THE INVENTION

The present invention teaches a customizable platform for ingredient selectable skin treatments. The customization platform, enables end users to purchase customized, fresh, and potent skin treatments and enables the end users to select the ingredients that resonate and work best for them. The skin care treatment product is shipped out for DIY mixing by the end user, avoiding longer warehouse storage and providing a level of quality and performance that end users simply can't get elsewhere.

End users purchase pre-configured kits, which provide combinations of active ingredients that are already grouped together (in their pre-mixed state). Next, end users configure their kits' active ingredients in the following ways: First an end user builds their own formulas with an active ingredients selection process, allowing the end user to specify the ingredients to include; Second, the end user completes a skin profile questionnaire where the end user answers a series of questions regarding their skin type and preferences.

The questionnaire is optional to all users, users who purchased pre-configured kits, kits based on manual selection of active ingredients, or our recommendations, or haven't purchased yet.

In either case, the platform of the present invention analyzes user data to determine, create, and assemble the ideal skincare product for the end user. Skin care products are shipped in proprietary packaging that includes a base cream, active ingredients, and a spoon or other stirring device or apparatus for mixing. Upon receiving the kit, the end user mixes the cream together with the active ingredients to create a potent solution as illustrated by the box directions.

When an end user first receives the kit or packaging, the end user unboxes the ingredients, which typically are comprised of the base cream jar and selected active ingredients. The end user then pours all the active ingredients into the base cream jar, one at a time. Using the provided spoon or other stirring device or apparatus of the packaging and kit, the end user mixes all the ingredients in a circular motion for about a minute. Next the end user closes the base cream jar and allows it to sit for about ten minutes. After about ten minutes, the mixed skin care treatment product is ready for use.

### BRIEF DESCRIPTION OF THE DRAWINGS

The accompanying drawings, which are incorporated herein and form a part of the specification, illustrate the present invention and, together with the description, further serve to explain the principles of the invention and to enable a person skilled in the pertinent art to make and use the invention.

FIG. 1 is a flow chart illustrating the purchase process of the skin care treatment product of the present invention.

FIG. 2 is an image of the process for using the kit taught by the present invention comprised of customizable skin care treatment ingredients and packaging that is used for mixing the ingredients.

FIG. 3 is a flow chart illustrating the automated formula recommendations process for skin care treatment product customization.

### DETAILED DESCRIPTION OF THE INVENTION

In the following detailed description of exemplary embodiments of the invention, reference is made to the accompanying drawings (where like numbers represent like elements), which form a part hereof, and in which is shown by way of illustration specific exemplary embodiments in which the invention may be practiced. These embodiments are described in sufficient detail to enable those skilled in the art to practice the invention, but other embodiments may be utilized and logical, mechanical, electrical, and other changes may be made without departing from the scope of the present invention. The following detailed description is, therefore, not to be taken in a limiting sense, and the scope of the present invention is defined only by the appended claims.

In the following description, numerous specific details are set forth to provide a thorough understanding of the invention. However, it is understood that the invention may be practiced without these specific details. In other instances, well-known structures and techniques known to one of ordinary skill in the art have not been shown in detail in order not to obscure the invention. Referring to the figures, it is possible to see the various major elements constituting the apparatus and method of the present invention.

The present invention is a customizable platform for ingredient selectable skin treatments in combination with a packaging system for mixing by an end user. The present invention reduces the lack of product potency and freshness that end users have been forced to deal with for way too long.

The present invention teaches a customizable platform for ingredient selectable skin treatments. The customization platform, enables end users to purchase customized and potent skin treatments and enables the end users to select the ingredients that resonate and work best for them.

The skin care treatment product is shipped out for DIY mixing by the end user, avoiding warehouse storage and providing a level of quality and performance that end users simply can't get elsewhere.

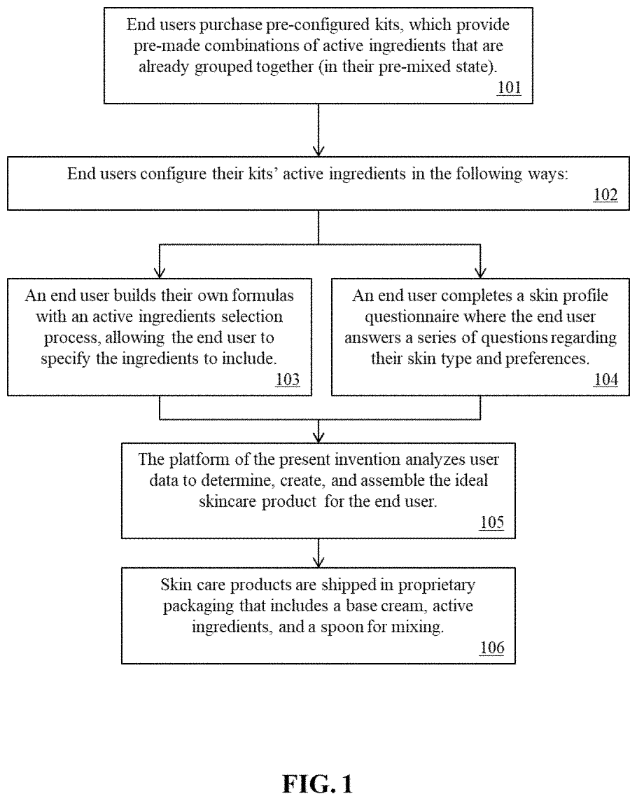

Now referring to FIG. 1, the purchase method for end users is illustrated. End users purchase pre-configured kits, which provide pre-made combinations of active ingredients that are already grouped together (in their pre-mixed state).

Next, end users configure their kits' active ingredients in the following ways: First an end user builds their own formulas with an active ingredients selection process, allowing the end user to specify the ingredients to include; Second, the end user completes a skin profile questionnaire where the end user answers a series of questions regarding their skin type and preferences.

The questionnaire is optional to all users, users who purchased pre-configured kits, kits based on manual selection of active ingredients, or our recommendations, or haven't purchased yet.

The questionnaire is used to determine our recommendation using data from external data sources. The present invention then collects customer satisfaction data which is used in correlation with an internal data source (questionnaire and/or skin photos as further explained) in order to make future recommendations

Future embodiments of the present invention plan to launch with different active ingredients, in an exemplary embodiment the current list is: Retinol, Collagen, Hyraulonic Acid, Oxygen, Vitamin C, AHA, Caviar, Placenta, DNA, Azulene, Chamoille, Stem Cell, Peptides, Vitamin E, Vitamin B, Vitamin D, Argan Oil. A do not mix together list includes: Retinol+Hydroxy Acids, Retinol+Vitamin C, and Retinol+AHA.

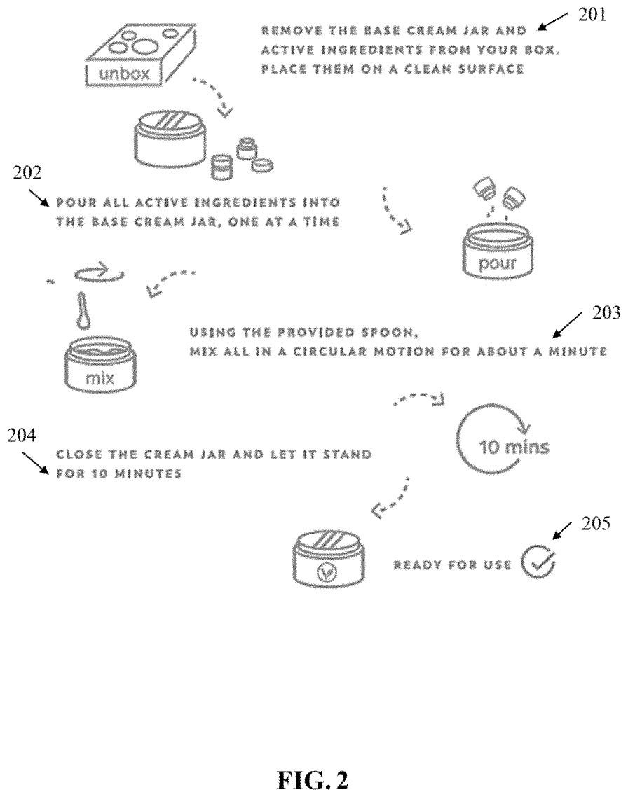

In either case, the platform of the present invention analyzes user data to determine, create, and assemble the ideal skincare product for the end user. Skin care products are shipped in proprietary packaging that includes a base cream, active ingredients, and a spoon or other stirring device or apparatus for mixing as illustrated by FIG. 2. Upon receiving the kit, the end user mixes the cream together with the active ingredients to create a potent, tailored solution as illustrated by the box directions shown in FIG. 2. The solution is tailored if it was customized by the user otherwise it is pre-configured and not specifically tailored to the user.

Now referring to FIG. 2, when an end user first receives the kit or packaging, in a first step 201 the end user unboxes the ingredients, which typically are comprised of the base cream jar and selected active ingredients. In a second step 202 the end user then pours all the active ingredients into the base cream jar, one at a time. In a third step 203, using the provided spoon or other stirring device or apparatus of the packaging and kit, the end user mixes all the ingredients in a circular motion. In a fourth step 204, the end user closes the base cream jar and allows it to sit for approximately about ten minutes. Finally, in a fifth step 205 after about ten minutes, the mixed skin care treatment product is ready for use.

Product customization is performed in one of two manners, manual and automated. In the manual customization and selection process, selecting the active ingredients to be included in the skin care treatment product cream is done using any terminal such as the Internet, a website, or a software application.

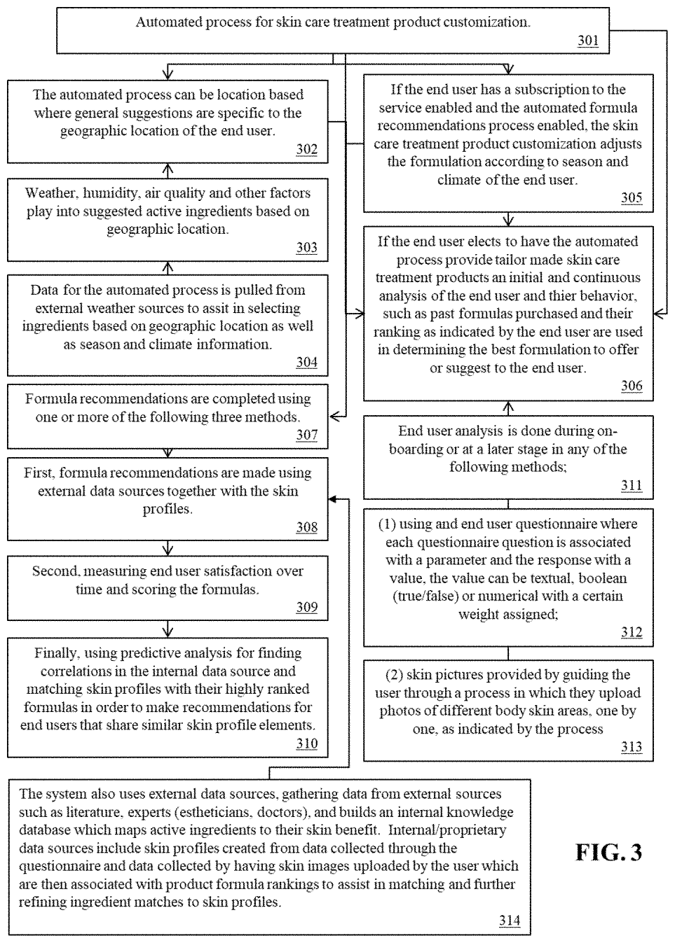

Now referring to FIG. 3, the automated formula recommendations process for skin care treatment product customization 301 is illustrated. The automated formula recommendations process is location based where general suggestions are specific to the geographic location of the end user 302. Weather, humidity, air quality and other factors play into suggested active ingredients based on geographic location 303.

The automated formula recommendations process takes the questionnaire data, which includes the location of the user, and recommends a formula by correlating it with external data sources for weather, humidity etc. and/or literature, industry experts such estheticians, doctors etc. 304.

If the end user has a subscription to the service enabled and the automated formula recommendations process enabled, the skin care treatment product customization can adjust the formulation according to season and climate of the end user 305. Data for the automated formula recommendations process is pulled from external weather sources to assist in selecting ingredients based on geographic location as well as season and climate information.

The subscription is for the delivery of creams every month for a fixed number of months. The product recommendations are not subscription dependent, it is a flag that the customer has set on their account which is set to true by default.

The system of the present invention will gather user satisfaction regardless of the automatic recommendations flag. An initial and continuous analysis of the end user and their behavior, such as past formulas purchased and their ranking as indicated by the end user are used in determining the best formulation to offer or suggest to the end user 306. End user analysis is done for all users. Product rankings will be collected from all users.

End user analysis 311 is done during on-boarding or at a later stage in any of the following methods; (1) using an end user questionnaire where each questionnaire question is associated with a parameter and the response with a value, the value is textual, Boolean (true/false) or numerical with a certain weight assigned 312; or (2) skin pictures provided by guiding the user through a process in which they upload photos of different body skin areas, one by one, as indicated by the process 313.

The initial recommendations will be done by correlating external data sources with the skin profiles. The continuous analysis will then track the satisfaction rate of those formulas. The data will then be used in order to recommend formulas to users who share similar profile attributes.

The system also uses external data sources, gathering data from external sources such as literature, experts (estheticians, doctors), and builds an internal knowledge database which maps active ingredients to their skin benefit. Internal/proprietary data sources include skin profiles created from data collected through the questionnaire and data collected by having skin images uploaded by the user which are then associated with product formula rankings to assist in matching and further refining ingredient matches to skin profiles 314.

Formula recommendations are completed using one or more of the following three methods 307. First, formula recommendations are made using external data sources together with the skin profiles 308. Second, measuring end user satisfaction over time and scoring the formulas 309. Finally, using predictive analysis for finding correlations in the internal data source and matching skin profiles with their highly ranked formulas in order to make recommendations for end users that share similar skin profile elements 310.

The questionnaire taught by the present invention includes the gather of general information such as: name, location, age, gender, ethnicity, skin concerns such as acne, dark spots, redness, wrinkles, oiliness, dryness, clogged pores, dullness, dark circles, not sure, other, brands liked, budget, and allergies such as none, nuts, gluten, acids, retinol, parabens, and fragrance. Finally, the questionnaire asks the end user to input a skin evaluation by asking a series of questions followed by listing a plurality of possible answers to select from for establishing an internal data source for matching skin profiles with highly ranked formulas and predicting formulas for new end users.

The questionnaire is optional to all users, users who purchased pre-configured kits, kits based on manual selection of active ingredients, or our recommendations, or haven't purchased yet.

The packaging design of the present invention includes detailed instructions on the box as shown in FIG. 2 on how to pour the active ingredients into the base cream jar. A custom made insert includes the base cream, active ingredients, and a spoon or other stirring device or apparatus for mixing.

The end user uses and experiences the product through the action of pouring all ingredients and creating the formula at home. The product is activated when mixed at home as previously discussed in the mixing description and illustrated in FIG. 2.

On the manufacturing side, the manufacturing and packaging departments receive skin care related formulations requests that include a base cream type in addition to one or more active ingredients to be included in the packaging. This information is then sent to a person or a robot to pick the ingredients and place them in a box. Finally, to further enhance product personalization the end users/customer's name can be affixed to the packaging for shipping.

In an alternative embodiment, with respect to specific formulations taught by the present invention, the present invention can be offered for sale in two pre-configured Blend kits, a Collagen Blend Cream and an Hyaluronic Acid Blend Serum. Each kit contains a fixed quantity of its ingredients and is delivered through the same customizable platform for ingredient selectable skin treatments in combination with a packaging system for mixing by an end user as also taught by the present invention.

In this alternative embodiment, the Blend kit is pre-configured for the end user with the manufacturer or distributor pre-selecting ingredients for the end user.

For a desired, pre-configured Collagen Blend cream, the skin treatment product has a chemical composition consisting of: Collagen: 1.75 ml; Vitamin: B 0.50 ml; Aloe Vera: 0.80 ml; Vitamin: A 0.80 ml; Vitamin: E 0.40 ml; Vitamin: C 0.80 ml; Peptide: 0.80 ml; Orange Essential Oil: 0.50 ml; and 60 ml of a base cream.

For a desired, pre-configured Hyaluronic Acid Blend serum, the skin treatment product has a chemical composition consisting of: Hyaluronic Acid: 1.70 ml; Aloe Vera: 0.80 ml; Apple Stem Cell: 0.80 ml; Vitamin A: 0.80 ml; Vitamin B: 0.50 ml; Vitamin E: 0.40 ml; Vitamin C: 0.80 ml; Rose Essential Oil: 0.60 ml; and 60 ml of a base cream.

It is appreciated that the optimum dimensional relationships for the parts of the invention, to include variation in size, materials, shape, form, function, and manner of operation, assembly and use, are deemed readily apparent and obvious to one of ordinary skill in the art, and all equivalent relationships to those illustrated in the drawings and described in the above description are intended to be encompassed by the present invention.

Furthermore, other areas of art may benefit from this method, and adjustments to the design are anticipated. Thus, the scope of the invention should be determined by the appended claims and their legal equivalents, rather than by the examples given.

1. A customizable platform for ingredient selectable skin treatments in combination with a packaging system for mixing by an end user comprising:
providing pre-configured kits, which provide combinations of active ingredients that are already grouped together in their pre-mixed state;
configuring a kits' active ingredients in the following ways:
enabling an end user to build their own formulas with an active ingredients selection process, allowing the end user to specify the ingredients to include;
enabling an end user to complete a skin profile questionnaire where the end user answers a series of questions regarding their skin type and preferences; and
the platform of the present invention analyzing the end user data to determine, create, and assemble the ideal skincare product for the end user.

2. The platform of claim 1, wherein the ingredients for a Collagen Blend Cream are pre-configured for the end user with respect to selectable skin treatments desired for or by the user, the ingredients selected from a group consisting of:
Collagen: 1.75 ml;
Vitamin: B 0.50 ml;
Aloe Vera: 0.80 ml;
Vitamin: A 0.80 ml;
Vitamin: E 0.40 ml;
Vitamin: C 0.80 ml;
Peptide: 0.80 ml;
Orange Essential Oil: 0.50 ml; and
60 ml of a base cream.

3. The platform of claim 1, wherein the ingredients for a Hyaluronic Acid Blend Serum are pre-configured for the end user with respect to selectable skin treatments desired for or by the user, the ingredients selected from a group consisting of:
Hyaluronic Acid: 1.70 ml;
Aloe Vera: 0.80 ml;
Apple Stem Cell: 0.80 ml;
Vitamin A: 0.80 ml;
Vitamin B: 0.50 ml;
Vitamin E: 0.40 ml;
Vitamin C: 0.80 ml;
Rose Essential Oil: 0.60 ml; and
60 ml of a base cream.

4. The platform of claim 1, wherein
skin care products are shipped in proprietary packaging that includes a base cream, active ingredients, and a spoon or other stirring device or apparatus for mixing.

5. The platform of claim 4, wherein
upon receiving the kit, the end user mixes the cream together with the active ingredients to create a potent, tailored solution as illustrated by the box directions.

6. The platform of claim 5, wherein
when an end user first receives the kit or packaging, the end user unboxes the ingredients, which typically are comprised of the base cream jar and selected active ingredients;
the end user then pours all the active ingredients into the base cream jar, one at a time;
using the provided spoon or other stirring device or apparatus of the packaging and kit, the end user mixes all the ingredients in a circular motion for about a minute;
the end user closes the base cream jar and allows it to sit for about ten minutes; and
after about ten minutes, the mixed skin care treatment product is ready for use.

7. The platform of claim 1, wherein
product customization is performed in one of two manners, manual and automated.

8. The platform of claim 7, wherein
in the manual customization and selection process, selecting the active ingredients to be included in the skin care treatment product cream is done using any terminal such as the Internet, a website, or a software application.

9. The platform of claim 7, wherein
in the automated formula recommendations process, the automated formula recommendations process is location based where general suggestions are specific to the geographic location of the end user.

10. The platform of claim 9, wherein
weather, humidity, air quality and other factors play into suggested base cream and active ingredients based on geographic location.

11. The platform of claim 7, wherein
if the end user has a subscription to the service enabled and the automated formula recommendations process enabled, the skin care treatment product customization adjusts the formulation according to season and climate of the end user.

12. The platform of claim 11, wherein
the subscription is for the delivery of creams every month for a fixed number of month; and
the product recommendations are not subscription dependent, it is a flag that the customer has set on their account which is set to true by default.

13. The platform of claim 11, wherein
data for the automated formula recommendations process is pulled from external weather sources to assist in selecting ingredients based on geographic location as well as season and climate information.

14. The platform of claim 7, wherein
an initial and continuous analysis of the end user and their behavior, such as past formulas purchased and their ranking as indicated by the end user are used in determining the best formulation to offer or suggest to the end user;
the continuous learning of user behavior via a formula satisfaction rate is done regardless of the product they purchase, pre-configured kits, manually configured kits via active ingredients selection or system recommendations;
data is collected from users in order to make recommendations to them or other users; and
if a user is satisfied with their formulas, the system will recommend similar formulas to other users that share similar skin profile attributes.

15. The platform of claim 14, wherein
end user analysis is done during on-boarding or at a later stage in any of the following methods;
using an end user questionnaire where each questionnaire question is associated with a parameter and the response with a value, the value is textual, Boolean (true/false) or numerical with a certain weight assigned; or
skin pictures provided by guiding the user through a process in which they upload photos of different body skin areas, one by one, as indicated by the process.

16. The platform of claim 15, wherein
the initial recommendations will be done by correlating external data sources with the skin profiles;
the continuous analysis will then track the satisfaction rate of those formulas; and
the data will then be used in order to recommend formulas to users who share similar skin profile attributes.

17. The platform of claim 15, wherein
the system also uses external data sources, gathering data from external sources such as literature, experts (estheticians, doctors), and builds an internal knowledge database which maps active ingredients to their skin benefit.

18. The platform of claim 15, wherein
internal/proprietary data sources include skin profiles created from data collected through the questionnaire and data collected by having skin images uploaded by the user which are then associated with product formula rankings to assist in matching and further refining ingredient matches to skin profiles.

19. The platform of claim 15, wherein
formula recommendations are completed using one or more of the following three methods:
formula recommendations are made using external data sources together with the skin profiles;
measuring end user satisfaction over time and scoring the formulas; and
using predictive analysis by finding correlations in the internal data source and matching skin profiles with their highly ranked formulas in order to make recommendations for end users that share similar skin profile elements.

20. The platform of claim 15, wherein
the questionnaire taught by the present invention includes the gather of general information such as: name, location, age, gender, ethnicity, skin concerns such as acne, dark spots, redness, wrinkles, oiliness, dryness, clogged pores, dullness, dark circles, not sure, other brands liked, budget, and allergies such as none, nuts, gluten, acids, retinol, parabens, and fragrance; and
the questionnaire asks the end user to input a skin evaluation by asking a series of questions followed by listing a plurality of possible answers to select from for establishing an internal data source for matching skin profiles with highly ranked formulas and predicting formulas for new end users.
